(19) United States
(12) Patent Application Publication
(10) Pub. No.: US 2025/0332401 A1
(43) Pub. Date: Oct. 30, 2025

(54) CONTROL DEVICE FOR A BLOOD PUMP

(71) Applicant: Berlin Heart GmbH, Berlin (DE)

(72) Inventor: Michael Frischke, Berlin (DE)

(73) Assignee: Berlin Heart GmbH, Berlin (DE)

(21) Appl. No.: 19/191,231

(22) Filed: Apr. 28, 2025

(30) Foreign Application Priority Data
Apr. 30, 2024 (EP) 24 173 607.3

(51) Int. Cl. A61M 60/508; A61M 60/122; G16H 40/63

(52) U.S. Cl. CPC A61M 60/508; A61M 60/122; G16H 40/63; A61M 2205/18; A61M 2205/3327; A61M 2205/3368; A61M 2205/502; A61M 2205/8262

(57) ABSTRACT

Control units for blood pumps and methods to operate such control units are disclosed. A control unit may comprise a blood pump port and include an energy storage unit and an energy supply unit. A charging interface may be configured to receive electrical energy from an external electrical energy source and a charge management unit may be configured to receive a charge level of the energy storage unit and to supply the energy storage unit with electrical charging power received via the charging interface to charge the energy storage unit. A control unit comprises at least one operating parameter detection unit configured to determine an operating parameter of the control unit. A charge management unit is designed to set a value of a charging voltage, a value of a charging current, an upper charging limit, and/or an alarm signal threshold value using at least one of the operating parameters.

### CROSS-REFERENCE TO RELATED APPLICATION

This application claims priority to EP Application No. 24 173 607.3, filed on Apr. 30, 2024, the entire content incorporated herein by reference.

### BRIEF DESCRIPTION OF THE DRAWINGS

Exemplary embodiments of control units and methods of operating control units are explained below with reference to the figures. For this purpose, an overview of the figures is provided first.

FIG. 1 shows a control unit for a blood pump;

FIG. 2 shows a diagram of how the value of any charging power, which is used to charge the control unit's energy storage unit from FIG. 1, changes over time;

FIG. 3 shows an alternative embodiment for a blood pump control unit;

FIG. 4A shows an upper charging limit and an alarm signal threshold in the case of a blood pump being connected to the blood pump port of the control unit shown in FIG. 3;

FIG. 4B shows the upper charging limit and an alarm signal threshold in the case of a blood pump being connected to the blood pump port of the control unit shown in FIG. 3;

FIG. 5 shows a blood pump device, which includes a blood pump and the control unit from FIG. 1; and

FIG. 6 shows an exemplary embodiment of a method to operate a control unit for a blood pump.

### DETAILED DESCRIPTION

This disclosure relates to a control unit for a blood pump, a blood pump device comprising a blood pump and a corresponding control unit, as well as a method to operate a control unit for a blood pump.

To enable mobility amongst patients with electrically operated medical devices, such as ventricular assist devices (VADs), these are often equipped with energy storage units. Due to the possibility of realizing high energy densities, these energy storage units often include lithium-ion rechargeable batteries (batteries for short). Besides the advantage of high energy densities, lithium-ion batteries also have several disadvantages. It has been found that the service life of lithium-ion batteries decreases especially rapidly when frequently fully charged and discharged. This is illustrated by the following example.

If a lithium-ion battery is always charged to 100% (4.2 V end-of-charge voltage), it will last for approximately 1000 charging cycles until the battery cell's total capacity has dropped to about 80%. If, however, the same battery cell is only ever charged to 95% (approx. 4.1 V end-of-charge voltage), it will last for approximately 1600 charging cycles until the total capacity has dropped to 80%.

The thermal behavior of lithium-ion batteries when charging may also be problematic. For example, if a lithium-ion battery is defective, it may overheat when charging. Charging lithium-ion batteries at high temperatures can also cause battery defects. Furthermore, at high ambient temperatures, even charging a non-defective lithium-ion battery can result in significant power loss and thus heat development. This poses a security problem not only for the device's user but also for the user's immediate surroundings.

The task underlying the present disclosure is to show an approach that at least partially counteracts the challenges described above. This approach is realized by control units and methods of operating control units disclosed in this application.

In an example a control unit includes a blood pump port to establish an electrical connection between the control unit and the blood pump. Furthermore, the control unit includes an energy storage unit to provide electrical energy and an energy supply unit configured to provide electrical energy supplied by the energy storage unit to the blood pump port to operate the blood pump. In addition, the control unit comprises a charging interface configured to receive electrical energy from an external electric energy source and a charge management unit configured to receive a charge level from the energy storage unit and to provide the energy storage unit with the electrical charging energy received via the charging interface to charge the energy storage unit. Furthermore, the charge management unit is configured to perform at least one of the following three functions: (1) to set a charging voltage value and/or a charging current value for the charging energy provided to the energy storage unit, using the charge level and at least one of the operating parameters, and/or (2) to set an upper charging limit up to which the energy storage unit is charged, using at least one of the operating parameters, and/or (3) to set an alarm signal threshold value, depending on which an alarm signal is provided, using at least one of the operating parameters.

Particularly when the energy storage unit is permanently installed inside the control unit, the inventors have realized that the control unit's service life depends to a large extent on the wear of the energy storage unit. As described above, however, frequent charging and discharging of the energy storage unit, especially of lithium-ion batteries, can lead to a reduction in charging capacity. If the charging capacity falls below a certain value, this may result in significant restrictions when using the device. As a result, this can lead to the unit having a shorter overall service life. Furthermore, exothermic reactions can also occur when charging such an energy storage unit, which can pose a major safety risk to the user in such case the energy storage unit is permanently installed. The device described above is designed to counteract these negative effects.

If any critical situations are detected on the basis of the one or more detected operating parameters, it is possible to terminate or at least reduce charging the energy storage unit for a short time by adjusting the charging voltage and/or charging current.

Furthermore, by setting an upper charging limit and/or an alarm threshold based on detected operating parameters, it is possible to influence how the energy storage unit is charged and discharged such that the energy storage unit's service life continues for as long as possible while minimizing the impact on the unit's user.

Further possible embodiments of the control unit are described below.

In one embodiment, the energy storage unit may be an internal energy storage unit. In this case, the control unit includes a control unit housing that encloses the control unit. Permanently installing the energy storage unit allows the energy storage unit to save space.

In another embodiment of the control unit, the at least one operating parameter detection unit may comprise, in addition to or as an alternative to the optional features mentioned, a temperature sensor unit, which is configured to determine an ambient temperature at the temperature sensor unit's location as an operating parameter. In this embodiment, the charge management unit can additionally be configured to set the value of the charging voltage and/or the value of the charging current, depending on the ambient temperature. This embodiment can be advantageous in preventing the control unit from overheating while the energy storage unit is being charged. This can increase safety for the user of the control unit.

In a variant of this embodiment, the interdependency between the charging power value and the ambient temperature can be selected such that the charging power value is lower the higher the ambient temperature is. This feature can help to prevent a critical temperature from being exceeded at or within the control unit.

In another variant, in addition to or as an alternative to the above-described feature, the temperature sensor unit can be arranged in the interior of the control unit, in close proximity to a surface of the control unit's housing and/or in close proximity to the electrical energy storage unit. These arrangements of the temperature sensor unit can be useful for monitoring the temperature of the control unit at critical points in and around the control unit.

In another exemplary embodiment of the control unit, the at least one unit to determine operating parameters may comprise, in addition to or as an alternative to the optional features of the above-described embodiments, a charging source detection unit, which is configured to determine as an operating parameter a charging source type of an external electrical energy source which supplies energy via the charging interface. Furthermore, the charge management unit in this embodiment can be configured to set the value of the charging voltage and/or the value of the charging current depending on the charging source type. This allows the charging voltage and/or charging current to be matched to the external energy source.

In a variant of this embodiment, the charging source detection unit can be configured to distinguish between a mains supply and a storage supply as charging source types. Furthermore, the relationship between the value of the charging current and/or charging voltage and the charging source type may be such that the value of the charging power is higher for a mains supply than for a storage supply.

In another embodiment, the at least one operating parameter detection unit may be additionally or alternatively configured to the features of the previously discussed optional embodiments, to provide power output data as an operating parameter, as a function of a power output delivered via the blood pump port. Furthermore, in this embodiment, the charge management unit can be configured to determine the upper charging limit and/or the alarm signal threshold depending on the power output data. This can be advantageous for keeping the charge level of the energy storage unit within a window, in which the energy storage unit's maximum capacity is only slightly lower by limiting the energy storage unit's charge and/or discharge. By adjusting the window to the power output data, the restriction on the energy storage unit's usable storage capacity can be adjusted at the same time so as to minimize any influence on the user. The energy storage unit's charge level indicates the amount of energy still remaining in the energy storage unit.

In a variant of this embodiment, the dependence between the upper charging limit and the power output data can be selected such that the lower the power supplied by the energy storage unit, the lower the charging limit. In addition or as an alternative to this, the dependence between the alarm signal threshold and the energy output data may be selected such that the higher the alarm signal threshold, the lower the power output of the energy storage unit. This choice of dependency can be advantageous to conserve the energy storage unit and at the same time to minimize the impact on the ability to use the control unit.

In another variant of this embodiment, the operating parameter detection unit providing the power output data can be configured to determine the power output data by measuring an electrical output, in addition to or as an alternative to the above-mentioned features. For example, measurements can be taken at the end of the blood pump, at the energy storage unit and/or at the charging interface.

In a further variant, the operating parameter detection unit that provides the power output data can be additionally or alternatively configured to provide the power output data depending on whether a blood pump is connected to the blood pump port. Setting the power output like this can be particularly easy to implement.

In a further variant of this embodiment, in addition to or as an alternative to the features of the previously described variants, the charge management unit can be configured to set the alarm signal threshold and/or the upper charging limit depending on the maximum capacity of the energy storage unit and of the power output data.

In the above-described case, the higher alarm signal threshold is an upstream alarm to enable the control unit to be operated in a battery saving manner. Additionally or alternatively, the charge management unit can also be configured to set the alarm signal threshold using the power output data and the control unit's given minimum remaining operating time. The alarm signal threshold determined like this would then correspond to an alarm signal threshold for an emergency alarm.

In another embodiment, in addition to or as an alternative to the features of the other optional embodiments, the at least one operating parameter detection unit can comprise an energy storage status detection unit configured to provide energy storage status data as an operating parameter depending on an energy storage unit's status. Furthermore, in this embodiment, the charge management unit can be configured to set the upper charging limit and/or the alarm signal threshold and/or the charging voltage and/or the charging current depending on the energy storage status data. Taking into account the charging status of the energy storage unit when setting the upper charging limit and/or alarm signal threshold and/or charging voltage and/or charging current can be advantageous in minimizing any use restrictions on the control unit.

In one variant of this embodiment, the energy storage status data can include a current maximum capacity for the energy storage unit. Furthermore, the charge management unit can be configured to set the upper charging limit and/or the alarm signal threshold relative to the maximum capacity at present. Additionally or alternatively, the energy storage status data can include a storage health status and the charge management unit can be configured to set the value of the charging power depending on the storage health status.

In another embodiment of the control unit, in addition to or as an alternative to the features of the above-described optional embodiments, the at least one operating parameter detection unit can comprise an input interface configured to determine the operating parameter based on a user input. Additionally, in this embodiment, the charge management unit can be configured to set the upper charging limit and/or the alarm signal threshold and/or the charging voltage and/or the charging current, depending on the user input. The option of allowing the user to select the upper charging limit and/or the alarm signal threshold and/or the charging voltage and/or the charging current can make it possible to adapt the control unit more closely to the user's requirements.

In a further embodiment, in addition to or as an alternative to the features of the previously described embodiments, the charge management unit can be configured to only provide the electric charging power to the energy storage unit as long as the charge level is below the upper charging limit, and/or the charge management unit can be configured to provide the alarm signal when the charge level drops below the alarm signal threshold.

In another embodiment, the charge management unit can additionally or alternatively be configured to set the value of the charging voltage and/or the charging current by selecting from a plurality of predefined value levels depending on the at least one operating parameter.

In another embodiment, the charge management unit can additionally or alternatively be configured to set the value of the charging voltage and/or the charging current such that this varies between two extreme values over time, depending on the at least one operating parameter. This approach corresponds to pulse width modulation and is one way of adjusting an average charging voltage and/or a charging current depending on the at least one operating parameter.

In variants of this embodiment, the charge management unit can be configured to periodically vary the value of the charging power, preferably in the form of a rectangular function, and to determine a dwell time of the value of the charging power at the respective charging power extremum as a function of the at least one operating parameter.

In another embodiment, the charging interface may be a charging connection. The charging connection may include a plug connector that allows the external energy source to be connected via a charging cable. This may be provided, for example, when the control unit is used as an extracorporeal control unit, i.e. a control unit that is outside the patient's body during use. The control unit is connected by means of a connection cable (a driveline) that is passed through a puncture site in the skin into the patient's body, electrically connected to an implantable blood pump.

In an alternative embodiment, the charging interface can be configured to receive electrical energy wirelessly. In this case, the control unit may be, for example, an implantable control unit. The charging interface allows the implanted control unit to receive electrical energy from a transcutaneous energy transfer (TET) device through the patient's tissue. The charging interface can, for example, be configured to receive electrical energy via induction.

In another embodiment, additionally or alternatively to the above-described features in connection with the other embodiments, the control unit can be a component of a blood pump device which, in addition to the control unit, also comprises a blood pump and preferably a connecting cable (driveline) to establish an electrical connection between blood pump and control unit.

In another embodiment, the energy storage unit may be a battery. In variants of this embodiment, the battery may be a lithium-ion battery.

A method to operate a control unit for a blood pump may comprise the following steps: supplying electrical energy via an energy storage unit in the control unit to a blood pump port in the control unit to operate the blood pump; receiving electrical energy at the control unit's charging interface and providing the electrical energy as charging power to the energy storage unit to charge the energy storage unit; and determining at least one operating parameter of the control unit.

Such a method includes at least one of the following method steps: setting a charging voltage value and/or a charging current value for the charging power supplied to the energy storage unit, using a charge level of the energy storage unit and at least one of the operating parameters; and/or setting an upper charging limit up to which the electrical energy storage unit is charged, using at least one of the operating parameters; and/or setting an alarm signal threshold value, depending on which an alarm signal is provided, using at least one of the operating parameters.

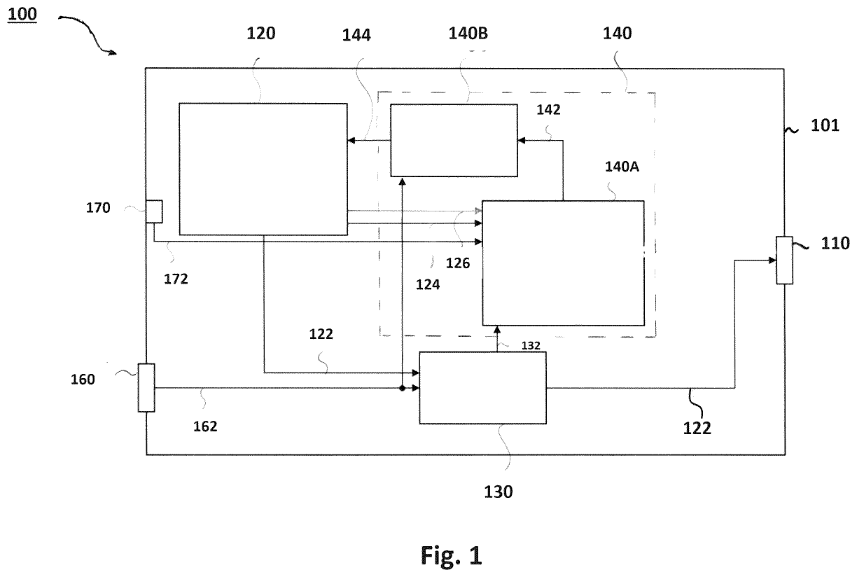

The following describes in detail the exemplary embodiment shown in the figures. An initial exemplary embodiment is described first on the basis of FIGS. 1, 2 and 5.

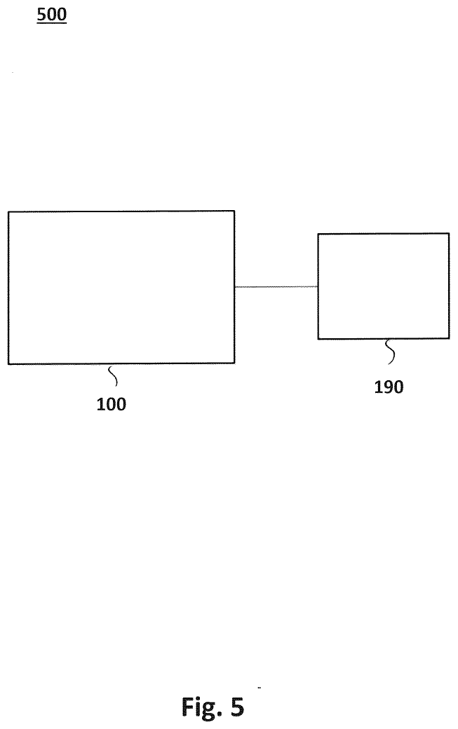

FIG. 1 shows a control unit 100 for a blood pump 190. FIG. 5 shows a blood pump device 500, which comprises the control unit 100 together with the blood pump 190.

The control unit 100 is configured to provide electrical energy to operate the blood pump 190. For this purpose, the control unit 100 includes a blood pump port 110 to establish an electrical connection between the control unit 100 and the blood pump 190, an energy storage unit 120 to supply electrical energy and an energy supply unit 130. The energy supply unit 130 is configured to receive electrical energy 122 supplied by the energy storage unit 120 to operate the blood pump 190 and to make this available at the blood pump port 110. In the example of the control unit shown in FIG. 1, the energy storage unit 120 is a lithium-ion battery. In principle, however, any other battery can also be used with the control unit.

Furthermore, the control unit 100 comprises a charging connection 160 as a charging interface through which, via a charging cable, the control unit 100 can be connected to an external electrical energy source and a charge management unit 140. The charge management unit 140 is designed to receive a charge level 124 from the energy storage unit 120 and to supply the energy storage unit 120 with electrical energy 162 received via the charging connection 160 to charge the energy storage unit 120. The charge management unit 140 can be implemented in different ways. In the exemplary embodiment shown in FIG. 1, the charge management unit 140 comprises a microcontroller 140A and a charging circuit 140B. The microcontroller 140A is configured to receive the charge level 124 of the energy storage unit 120 and to set a value 142 of a charging power 144 supplied to the energy storage unit 120 and to transmit it to the charging circuit 140B. In turn, the charging circuit 140B is configured to receive the value of the charging power 142 and energy supplied to the charging connection 160 and to supply the energy storage unit 120 accordingly with charging power 144.

The control unit 100 shown in FIG. 1 is also able to send control signals to the blood pump 190. In some embodiments, the microcontroller 140A can include all functional units necessary for supplying the charging power and for providing the control signals. In other exemplary embodiments, however, the function groups can also be accommodated in different microcontrollers.

Furthermore, the control unit 100 comprises several operating parameter detection units that are configured to determine an operating parameter of the control unit 100. In addition, the charge management unit 140 is configured to set a value 142 of the charging power for the charging power 144 supplied to the energy storage unit 120 using the charge level 124 and at least one of the operating parameters.

In the exemplary embodiment shown in FIG. 1, the operating parameter detection unit is a temperature sensor unit 170 configured to detect an ambient temperature 172 at the location of the temperature sensor unit 170 as an operating parameter.

In the exemplary embodiment shown, the temperature sensor unit 170 is arranged between the energy storage unit 120 and a housing surface of a housing 101 of the control unit 100 closest to the energy storage unit 120. Furthermore, to prevent the control unit's surface from exceeding a critical temperature, the interdependence between the value 142 of the charging power and the ambient temperature 172 is selected such that the lower the value 142 of the charging power, the higher the ambient temperature 172. The arrangement, however, of the temperature sensor unit 170 shown is only an example. In other embodiments, the temperature sensor unit may also be located at a different location within or on the control unit.

In addition to the temperature sensor unit 170, the energy supply unit 130 includes a charging source detection unit as a further operating parameter detection unit, which is configured to determine a charging source type 132 of an electrical energy source connected to the charging connection 160 as a further operating parameter. Furthermore, the charge management unit 140 is configured to set the value 142 of the charging power depending on the charging source type 132. There are several ways to determine the charging source type. In the embodiment shown, the energy supply unit 130 is configured to determine the charging source type 132 according to a charging voltage supplied to the charging connection.

In the exemplary embodiment, the charging source detection unit can, for example, distinguish via the charging connection 170 whether the control unit 100 is connected to a mains supply or to an external energy storage unit. Furthermore, the microcontroller 140A is configured to set the value of the charging power depending on the charging source type 132 received. This allows the charging source to provide appropriate charging power 144 to charge the energy storage unit, for example, a higher charging capacity when connected to the grid than when connected to an external energy storage unit. When connecting an external energy storage unit, it is often also advantageous to initially use most of the power supplied by the external energy storage unit to operate the blood pump. This can prevent the external energy storage unit from emptying quickly, prompting the user to replace the external battery after a short time. These considerations can also be incorporated into the microcontroller 140A to implement a dependence between charging source type 132 and the value 142 of the charging power.

In addition, in the exemplary embodiments shown in FIG. 1, the energy storage unit 120 itself comprises an energy storage status detection unit as a further operating parameter detection unit, which is configured to provide energy storage status data 126 as an operating parameter, depending on a status of the energy storage unit 120. In addition, the microcontroller 140A of the charge management unit 140 is configured to set the value 142 of the charging power depending on the energy storage status data 126. The energy storage status data 126 includes a memory health status, which is an indicator of wear on the energy storage unit 126. Furthermore, the microcontroller 140A of the charge management unit 140 is configured to select the value 142 of the charging power to be all the lower, the worse the storage health status.

In the control unit 100 shown in FIG. 1, the value of the charging power is set in dependence of the charge level 124 and at least one of the operating parameters 126, 132, 172. The microcontroller 140A can be configured to increase or decrease the charging power in accordance with the operating parameters. This can be done, for example, by selecting from a variety of predefined value levels depending on one or more operating parameters.

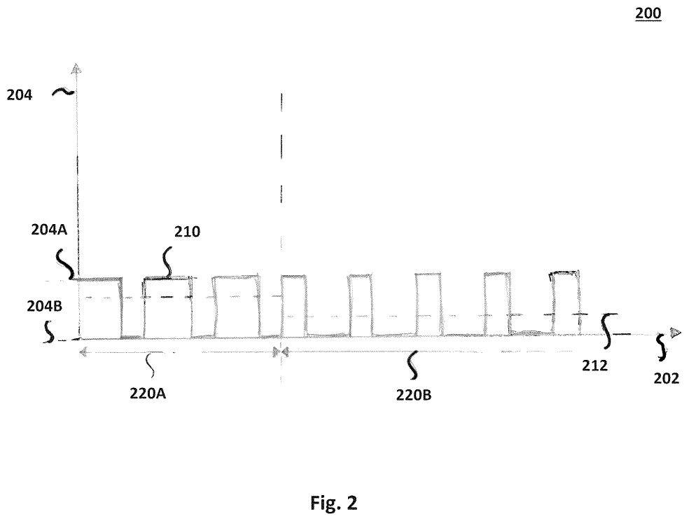

Alternatively, however, it is also possible to specify the charging power using pulse width modulation. This is explained in greater detail below with reference to FIG. 2.

FIG. 2 shows a diagram 200 of how the value of any charging power which is used to charge an energy storage unit 120 of the control unit from FIG. 1 changes over time 210.

In diagram 200, how the value of the charging power changes over time is shown on an amplitude axis 204 as a function over a time axis 202. The value of the charging power varies over time in the form of a rectangular function between a maximum value of 204A and a minimum value of 204B, which in this case is zero. The diagram shows that in the first time period of 220A, the value of the charging power is on average for ⅔ of the time of a period of the rectangular function at the maximum value 204A and only in ⅓ of the time at the minimum value 204B. The mean value of the amplitude is 212 ⅔ of the maximum value 204A. In a second time period, 220B, the value of the charging power is on average only ⅓ of the maximum value. Depending on the choice of the time durations at which the charging power is at the maximum or minimum value, any average charging power between the maximum and minimum value can be set.

In the exemplary embodiment described here, the value 142 for the charging power is set by the charge management unit 140. A value for either a charging voltage or a charging current may however be specified in other exemplary embodiments and only the corresponding other value may vary.

In the example shown of control unit 100, various environmental parameters are used in conjunction to set the value of 142 for the charging power. In principle, however, it is also possible to use any selection of these or any other environmental parameters.

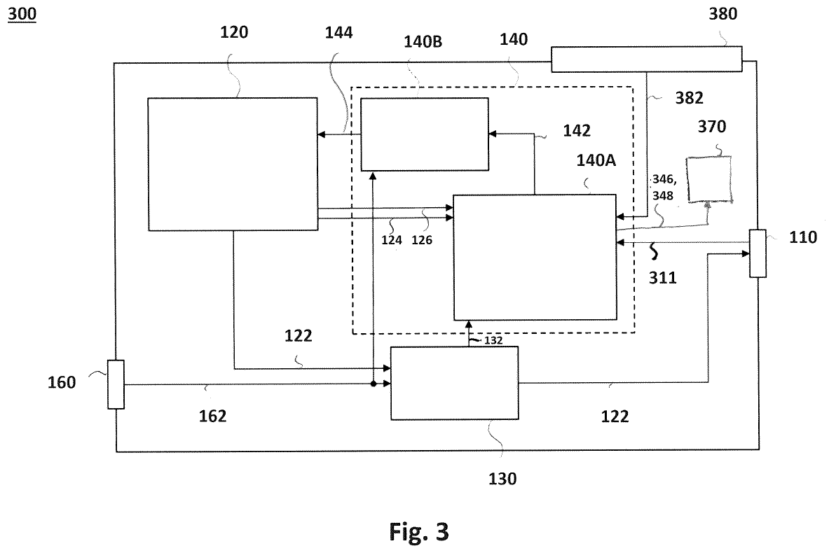

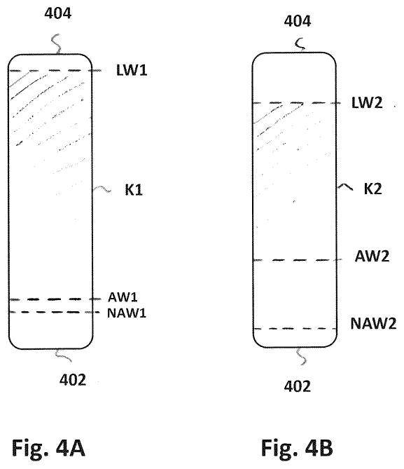

Alternatively or in addition to setting the charging power, environmental parameters can also be used to set other parameters relevant for charging the energy storage unit. Described below is a corresponding exemplary embodiment with reference to FIGS. 3 and 4.

FIG. 3 shows an alternative embodiment of a control unit 300 for a blood pump.

The exemplary embodiment shown in FIG. 3 is almost identical to the exemplary embodiment shown in FIG. 1. All components already shown in FIG. 1 are labelled with the same reference number in FIG. 3 and will only be described in the following if their mode of operation differs from the exemplary embodiment shown in FIG. 1.

One difference from the design example of FIG. 1 is that the charge management unit 140 of the control unit 300 is, using operating parameters, configured to set an upper charging limit up to which the energy storage unit 120 is charged and to set an alarm signal threshold value, depending on which an alarm signal 346 is provided using at least one of the operating parameters. The charge management unit 140 is further configured to supply the energy storage unit 120 with the electrical charging power only as long as the charge level of the energy storage unit 120 is below the upper charging limit. Furthermore, the charge management unit 140 is configured to provide the alarm signal 346 when the charge level of the energy storage unit 120 falls below the alarm signal threshold. In the specific embodiment of the control unit 300, the microcontroller 140A is configured to compare the charge level 124 of the energy storage unit 120 at regular intervals with the upper charging limit and the alarm signal threshold. If the charge level 124 exceeds the upper charging limit, the microcontroller 140A is configured to reduce the charging power 142 to zero, so that the charging circuit 140B no longer charges the energy storage unit 120. Should the charge level 124 fall below the alarm signal threshold, the microcontroller 140A is configured to provide the alarm signal 346. The alarm signal 346 is then forwarded to an alarm unit 370, which, depending on the alarm signal, outputs an audible warning signal 142. The output of the audible warning signal should only be understood as an example. In other exemplary embodiments, the warning signal may also be sent on to a user of the control unit in a different form. The upper charging limit and the alarm signal threshold can, for example, be based on a voltage, i.e. an end-of-charge voltage or an alarm signal threshold voltage of the energy storage unit 120.

It is known that repeatedly charging and discharging certain types of energy storage units, such as lithium-ion batteries, can lead to a reduction in the storage capacity of the electrical energy storage unit 120. This should be counteracted by setting the upper charging limit and the alarm signal threshold value in dependence on the environmental parameters.

A general reduction of the upper charging limit of the energy storage unit 120 and a general increase of the alarm signal threshold for the energy storage unit 120 can be used to prevent the energy storage unit 120 from always being fully charged or almost fully discharged. This can reduce the service life of the energy storage unit 120. This has a major negative impact, however, on the usability of the blood pump's control unit 300 because the control unit 300 then requires more frequent charging. To counteract this negative influence, the control unit 300 comprises a plurality of operating parameter detection units to reduce the upper charging limit and/or increase the alarm signal threshold only if this only slightly influences the use of the control unit.

One situation in which any restriction on charging or discharging of the energy storage unit 120 has little effect on the user is when either the blood pump is not connected to the blood pump port 110 at all or the blood pump's required electrical power is low. This may be the case, for example, if the control unit is acting as a replacement device and is only used when a control unit in current use needs replacing, due to a defect for example. To adjust the upper charging limit and alarm signal threshold only at moments when the power supply to the blood pump charge port is low, the control unit 300 includes a blood pump port 110 with an operating parameter detection unit configured to determine whether a blood pump is connected to the blood pump port and, depending on this, to provide power output data 110 to the charge management unit. The charge management unit 140 is further configured to set both the upper charging limit as well as the alarm signal threshold as a function of the power output data. A resistance or voltage measurement can be used, for example, to set whether a blood pump is connected to the blood pump port 110. It is also possible to use a separate pin to detect the blood pump.

In other exemplary embodiments, the energy supply unit 130 may, as an alternative or in addition, comprise an operating parameter detection device configured to determine an electrical power output at the blood pump port 110 and, depending on this, to provide the charge management unit 140 with the power output data.

When charging the energy storage unit 120, it may also be useful to take into account the type of external energy source used to charge the energy storage unit 120. If, for example, an energy storage unit is also connected to charging connection 160 and its service life is negatively affected by a complete discharge, it may be useful to also take the charging source type into account when setting the upper charging limit. As already described for the control unit 100, the energy supply unit 130 of the control unit 300 therefore includes a charging source detection unit, which is configured to detect a charging source type 132, as a further operating parameter, of an electrical energy source connected to the charging connection 160. Furthermore, the charge management unit 140 is configured to set the value 142 of the upper charging limit depending on the charging source type 132. The relationship between the charging source type and the upper charging limits can be configured in different ways. The upper charging limit is set lower if the external energy source is an external energy storage unit, as shown in the exemplary embodiment here. In other embodiments, the storage capacity of the external energy source can also be considered in addition to this, or a distinction can be made between different types of external energy storage units.

Especially if the effective capacity of the energy storage unit 120 is reduced by setting a lower charging limit and/or a higher alarm signal threshold, the user should also have the possibility to influence these settings. This is useful, for example, if the user already knows that a longer excursion is planned and that it may not be possible to charge the energy storage unit 120 for a certain period of time. For such cases, the control unit 300 also includes an input interface 380 as an additional operating parameter detection unit, which is configured to determine an operating parameter 382 on the basis of a user input. Additionally, depending on the user's input, the charge management unit 140 is configured to set the upper charging limit and the alarm signal threshold. For example, user input can be made by selecting an operating mode. For example, there could be an eco mode, which would involve reduced charging and discharging of the energy storage unit 120, and a mobility mode, which utilizes the full storage capacity of the energy storage unit 120.

The following example, based on FIGS. 4A and 4B, describes how the upper charging limit and the alarm signal threshold are adjusted using the control unit 300.

FIG. 4A shows an upper charging limit and an alarm signal threshold in the case where a blood pump is connected to the blood pump port 110. FIG. 4B shows an upper charging limit and an alarm signal threshold in the case where no blood pump is connected to the blood pump port 110. FIGS. 4A and 4B show an illustration of the charge level of the energy storage unit 120, where a lower limit 402 represents a energy storage unit 120 being fully discharged and an upper limit 404 represents an energy storage unit 120 being fully charged. The upper charging limit and the alarm signal threshold can vary between these two limits.

As described above, depending on whether a blood pump is connected to the blood pump port 110, the blood pump port 110 is configured to provide power output data 311. If a blood pump is connected, it must be assumed that the power output to the blood pump will lead to a rapid reduction of the energy storage's charge level. To enable use of the control unit 300 over a longer period of time, the charge management unit 140 is configured to set the upper charging limit to a value LW1 and to set the alarm signal threshold to a value AW1, both of which are very close to the respective limits 402 and 404 of the energy storage unit 120. As illustrated by the shaded area, a large storage capacity K1 becomes available when the energy storage unit 120 is charged up to the upper charging limit LW1 and discharged to the alarm signal threshold AW1. However, because the upper charging limit CL1 is very close to the upper limit 404 and the alarm signal threshold AS1 is at the lower limit 402, repeatedly charging or discharging the energy storage unit 120 up to these limits can, over time, reduce the total storage capacity of the energy storage unit 120.

For this reason, if no blood pump is connected to the blood pump port 110, the charge management unit 140 is configured to lower the upper charging limit to a value LW2 and to raise the alarm signal threshold to a value AW2. This is illustrated in FIG. 4B. This leads to a reduced usable capacity K2. However, as the power output of the energy storage unit 120 is also reduced, the control unit 300 can be operated for a comparable period of time illustrated in the case of FIG. 4A, or perhaps even longer. At the same time, the energy storage unit is protected such that the storage capacity of the energy storage unit 120 is reduced less even by repeatedly charging and discharging.

The charge management unit 140 is configured to set the upper charging limit and the alarm signal threshold value depending on the maximum capacity of the energy storage unit 120, an expected wattage of the control unit 300, which can be determined from the power output data 311, and a minimum operating time. In some configurations of the control unit, the charge management unit 140 is additionally configured to take into account a status of the energy storage unit 120 when setting the upper charging limit and the alarm signal threshold. For example, the current maximum capacity of the energy storage unit can be taken into account when setting the upper charging limit and the alarm signal threshold, so that even if the maximum capacity is reduced due to wear, a usable capacity can be provided according to the power to be supplied at the blood pump port 110.

In addition to the alarm signal threshold already described, some embodiments may also be configured to set a threshold for an emergency alarm signal. It may be helpful for control unit's user to receive an additional audible warning signal if the wattage falls below a predefined minimum remaining time of the control unit.

The charge management unit 140 of the control unit 300 is therefore configured to not only set the upper charging limit and the alarm signal threshold but also a value for the emergency alarm signal threshold. This value is set such that the emergency alarm signal is triggered when the expected wattage of the control unit 300, which can be set from the power output data 311, is exceeded by a predefined minimum remaining time of the control unit 300. Furthermore, the charge management unit 140 is configured to provide the alarm unit 370 with the emergency alarm signal 348 when the charge level in the energy storage unit 120 falls below the emergency alarm signal threshold value. The alarm unit 370 is in turn configured to output a second audible warning signal in response to the emergency alarm signal 348.

In the example shown in FIG. 4A, the power output is high due to the blood pump being connected to the blood pump port. In the example shown in FIG. 4B, the power output is low due to no blood pump being connected to the control unit. In both cases, the minimum remaining time of the control unit is identical. However, due to the different power outputs, the emergency alarm signal threshold NAW1 in FIG. 4A set by the charge management circuit 140 is higher than the emergency alarm signal threshold NAW2 set in FIG. 4B.

In the exemplary embodiments of FIG. 1, the charging power's value is set using at least one operating parameter. In the exemplary embodiment of FIG. 3, the upper charging limit and the alarm signal threshold are set using at least one operating parameter. However, this is only an example. In other embodiments, the control unit can be used to select any of these three variables, i.e. only one of the four variables, several of the four variables or all four variables.

Furthermore, a control unit is shown in FIGS. 1 and 3 that is worn outside the patient's body and can be connected by cable to an external energy source via the charging connection. However, it is also possible for the control unit to have a charging interface instead of a charging connection with a plug connector, which is configured to receive wireless electrical energy via an inductive transfer of energy. Such a control unit can also be configured to be implantable, meaning it can be implanted in the patient's body together with the blood pump and receive electrical energy from a charging device outside the body via a transcutaneous energy transfer (TET). In this embodiment, the charging source type can be detected, for example, by the energy supplying device also providing corresponding charging source type data via the charging interface. In this case, a unit for recording environmental parameters of the control unit can be configured to determine a charging source type of the external energy source using charging source type data.

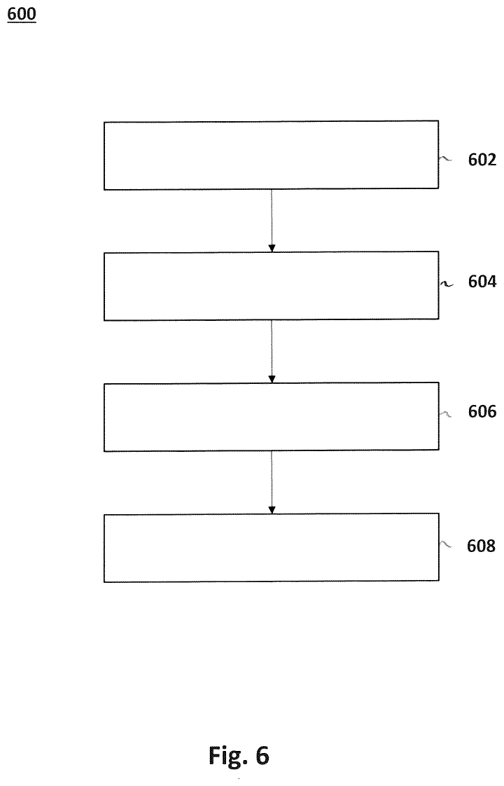

Finally, a method for operating a control unit for a blood pump is described with respect to FIG. 6.

FIG. 6 shows individual method steps 602-608 of a method 600 to operate the control unit.

The method 600 begins with a method step 602, which comprises the supply of electrical energy to a blood pump port of the control unit via an energy storage unit in the control unit for operating the blood pump.

A second method step 604 involves receiving electrical energy at a charging interface of the control unit and providing the energy storage unit with electrical energy in the form of charging power to charge the energy storage unit.

Another method step 606 involves determining at least one operating parameter of the control unit.

In one method step 608, the following are optional: setting a charging voltage and/or a value of a charging current for the charging power provided at the energy storage unit, using a charge level of the energy storage unit and at least one of the operating parameters; setting an upper charging limit up to which the electrical energy storage unit is charged, using at least one of the operating parameters; and/or setting an alarm signal threshold value, depending on which an alarm signal is provided, using at least one of the operating parameters.

As already explained according to the exemplary embodiments of the control unit 100 and 300, only one of the variables charging voltage, charging current, upper charging limit or alarm signal threshold can be set in method step 608, several of these variables or all of these variables can be set.

In summary, this disclosure relates to a control unit (100) for a blood pump (190), comprising a blood pump port (110) to establish an electrical connection between the control unit (100) and the blood pump (190). Additionally, the control unit (100) includes an energy storage unit (120) to supply electrical energy and an energy supply unit (130) that is configured to provide electrical energy supplied by the energy storage unit (120) to the blood pump port (110) to operate the blood pump (190). Furthermore, the control unit (300) comprises a charging interface (160) that is configured to receive electrical energy from an external electrical energy source, and a charge management unit (140) configured to receive a charge level (124) of the energy storage unit (120) and to supply electrical charging power received via the charging interface (160) to the energy storage unit (120) to charge the energy storage unit (120). Additionally, the control unit (100) comprises at least one operating parameter detection unit (170) that is configured to determine an operating parameter (172) of the control unit (100). Furthermore, the charge management unit (140) is configured to set a value (142) of a charging voltage, a value (142) of a charging current, an upper charging limit (LW1, LW2) and/or an alarm signal threshold value (AW1, AW2) using at least one of the operating parameters (172).

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one or more element alone or the one or more element in combination with one or more of the other elements which may also include, in combination, additional elements not listed. Unless otherwise indicated or the context suggests otherwise, as used herein, “a” or “an” means “at least one” or “one or more.”

1. A control unit for a blood pump, comprising:
a blood pump port to establish an electrical connection between a control unit and a blood pump;
an energy storage unit to supply electrical energy;
an energy supply unit configured to provide the blood pump port with electrical energy supplied by the energy storage unit to operate the blood pump;
a charging interface configured to receive electrical energy from an external electrical energy source; and
a charge management unit configured to receive a charge level of the energy storage unit and to supply electrical charging power received via the charging interface to the energy storage unit to charge the energy storage unit;
wherein at least one or more of the following:
the control unit comprises at least one operating parameter detection unit configured to determine an operating parameter of the control unit;
the charge management unit is configured to set a value of a charging voltage and/or a value of a charging current for the charging power supplied to the energy storage unit using the charge level and at least one of the operating parameters;
the charge management unit is configured to set an upper charging limit up to which the energy storage unit is charged, using at least one of the operating parameters; and
the charge management unit is configured to set an alarm signal threshold value, depending on which an alarm signal is provided, using at least one of the operating parameters.

2. The control unit of claim 1, wherein the at least one operating parameter detection unit may comprise a temperature sensor unit, which is configured to determine, as an operating parameter, an ambient temperature at the location of the temperature sensor unit, and the charge management unit is further configured to determine the value of the charging voltage and/or the value of the charging current depending on the ambient temperature.

3. The control unit of claim 1, wherein the at least one operating parameter detection unit comprises a charging source detection unit configured to determine, as an operating parameter, a charging source type of an external electrical energy source that supplies energy via the charging interface, and the charge management unit is configured to determine the value of the charging voltage and/or the value of the charging current depending on the charging source type.

4. The control unit of claim 1, wherein the at least one operating parameter acquisition unit is configured to provide, as an operating parameter, power output data as a function of power supplied through the blood pump port, and the charge management unit is configured to determine the upper charging limit and/or the alarm signal threshold depending on the power output data.

5. The control unit of claim 4, wherein the operating parameter detection unit providing the power output data is configured to determine the power output data by measuring electrical power.

6. The control unit of claim 4, wherein the operating parameter detection unit providing the power output data is configured to provide the power output data depending on whether a blood pump is connected to the blood pump port.

7. The control unit of claim 4, wherein the charge management unit is configured to set the alarm signal threshold value and/or to set the maximum charging limit as a function of a maximum capacity of the energy storage unit and the power output data.

8. The control unit of claim 7, wherein the charge management unit is configured to use the power output data to set the alarm signal threshold and a given minimum remaining operating time of the control unit.

9. The control unit of claim 1, wherein the at least one operating parameter detection unit comprises an energy storage status detection unit that is configured to provide, as an operating parameter, energy storage status data depending on a status of the energy storage unit, and the charge management unit is configured to set at least one or more of the following as a function of the energy storage status data: the upper charging limit; the alarm signal threshold; the charging voltage; and the charging current.

10. The control unit of claim 1, wherein the at least one operating parameter detection unit comprises an input interface that is configured to determine the operating parameter based on a user input, and the charge management unit is set to determine depending on the user input at least one or more of the following: the upper charging limit; the alarm signal threshold; the charging voltage; and/or the charging current.

11. The control unit of claim 1, wherein the charge management unit is configured to supply the energy storage unit with the electrical charging power only as long as the charge level is below the upper charging limit, and/or the charge management unit is configured to provide the alarm signal when the charge level falls below the alarm signal threshold value.

12. The control unit of claim 1, wherein the charge management unit is configured to set, depending on the at least one operating parameter, the value of the charging voltage and/or the charging current by selecting from a plurality of predefined value levels.

13. The control unit of claim 1, wherein the charge management unit is configured to set the value of the charging voltage and/or the charging current to vary time-dependently between two extreme values as a function of the at least one operating parameter.

14. A blood pump device comprising:
a blood pump; and
a control unit comprising:
a blood pump port to establish an electrical connection between the control unit and the blood pump;
an energy storage unit to supply electrical energy;
an energy supply unit configured to provide the blood pump port with electrical energy supplied by the energy storage unit to operate the blood pump;
a charging interface configured to receive electrical energy from an external electrical energy source; and
a charge management unit configured to receive a charge level of the energy storage unit and to supply electrical charging power received via the charging interface to the energy storage unit to charge the energy storage unit;
wherein at least one or more of the following:
the control unit comprises at least one operating parameter detection unit configured to determine an operating parameter of the control unit;
the charge management unit is configured to set a value of a charging voltage and/or a value of a charging current for the charging power supplied to the energy storage unit using the charge level and at least one of the operating parameters;
the charge management unit is configured to set an upper charging limit up to which the energy storage unit is charged, using at least one of the operating parameters; and
the charge management unit is configured to set an alarm signal threshold value, depending on which an alarm signal is provided, using at least one of the operating parameters.

15. A method of operating a control unit for a blood pump, comprising:
supplying electrical energy via an energy storage unit in the control unit of a blood pump port of the control unit to operate the blood pump;
receiving electrical energy at the control unit's charging interface and supplying the electrical energy as charging power to the energy storage unit to charge the energy storage unit;
determining at least one operating parameter of the control unit;
setting a value of a charging voltage and/or a value of the charging current for the charging power supplied at the energy storage unit, using a charge level of the energy storage unit and at least one of the operating parameters; and/or
setting an upper charging limit up to which the electrical energy storage unit is charged, using at least one of the operating parameters; and/or
setting an alarm signal threshold value, depending on which an alarm signal is provided, using at least one of the operating parameters.
